(19) United States
(12) Patent Application Publication
(10) Pub. No.: US 2026/0041389 A1
(43) Pub. Date: Feb. 12, 2026

(54) COMPUTER-IMPLEMENTED METHOD FOR OPERATING A MAMMOGRAPHY DEVICE, AND MAMMOGRAPHY DEVICE

(71) Applicant: Siemens Healthineers AG, Forchheim (DE)

(72) Inventor: Marcus RADICKE, Veitsbronn (DE)

(73) Assignee: Siemens Healthineers AG, Forchheim (DE)

(21) Appl. No.: 19/289,332

(22) Filed: Aug. 4, 2025

(30) Foreign Application Priority Data
Aug. 6, 2024 (EP) 24193104.7

(51) Int. Cl. A61B 6/50; A61B 6/00; A61B 6/04; A61B 6/46; A61B 90/00

(52) U.S. Cl. CPC A61B 6/502; A61B 6/0414; A61B 6/469; A61B 6/545; A61B 90/06; A61B 2090/064

(57) ABSTRACT

One or more example embodiments relates to a computer-implemented method for operating a mammography device, the method includes determining a reference surface value which describes an assumed contact surface between a compressed examination object and a movable compression paddle in a target compression setting, using the force value and the position value at at least one reference time instant; and compressing the examination object by actuating an actuator until a termination condition is fulfilled, wherein the termination condition describes the attainment of a predetermined setpoint pressure by the force value in relation to the reference surface value.

### CROSS-REFERENCE TO RELATED APPLICATION(S)

The present application claims priority under 35 U.S.C. § 119 to European Patent Application No. 24193104.7, filed Aug. 6, 2024, the entire contents of which are incorporated herein by reference.

### FIELD

One or more example embodiments relates to a computer-implemented method for operating a mammography device. One or more example embodiments also relates to a mammography device.

### RELATED ART

Mammography devices are X-ray imaging devices which typically comprise an object holder, on which an examination object, in particular a breast (mamma) can be positioned for acquisition with an acquisition arrangement. In order to irradiate as little overlapping tissue as possible, the examination object is compressed via a compression device between the object holder and a movable compression paddle. Here, the object holder can be or comprise the X-ray detector.

Compression in mammography offers many advantages, for example the reduction of movements, tissue overlap and average X-ray dose, better exploitation of the detector's spatial resolution, the reduction of scattered radiation and radiation hardening, and better utilization of the detector's dynamic range on account of the even thickness. It is nevertheless problematic that too strong a compression could be set, which can lead to discomfort and in some cases even pain.

A white paper by Johannes Georg Korporaal, “Breast Compression with SoftSpeed and OpComp”, Siemens Healthcare GmbH, 2017, describes a solution approach in which the change in breast thickness over time, which can be set in relation to an effective compression force, is analyzed and a threshold value defined for the gradient of the change in thickness over time at which the movement of the compression paddle is stopped and which is used as the target compression setting.

### SUMMARY

It is however problematic in this context that individual differences exist in the breasts and, for this reason, different sensitivities with respect to the compression applied can occur when a threshold value for the gradient is used. Even a readjustment by an operator cannot always make any change thereto, as said operator is likewise unable to ascertain a perfect setting.

One or more example embodiments specifies a possibility for controlling the automatic movement of a compression paddle of a mammography device which enables improved comfort and improved identification of a suitable target compression setting.

This is achieved with a computer-implemented method and a mammography device as claimed in the independent claims. Advantageous developments are disclosed in the dependent claims.

### BRIEF DESCRIPTION OF THE DRAWINGS

Advantages and details of the present invention are disclosed in the exemplary embodiments described below and by reference to the drawing, in which:

FIG. 1 shows a flow diagram of an exemplary embodiment of the method according to the invention,

FIG. 2 shows a first possible display on a display device,

FIG. 3 shows a second possible display on a display device,

FIG. 4 shows a third possible display on a display device,

FIG. 5 shows a block diagram of a mammography device of an exemplary embodiment according to the invention, and

FIG. 6 shows the functional structure of a control device of the mammography device according to an exemplary embodiment.

### DETAILED DESCRIPTION

In a method of the type mentioned in the introduction, provision is made according to one or more example embodiments for the method to have the following steps for automatically adjusting a compression of the examination object for an acquisition process:
determining a reference surface value which describes an assumed contact surface between the compressed examination object and the movable compression paddle in the target compression setting, using the force value and the position value at at least one reference time instant, and
compressing the examination object by actuating the actuator until a termination condition is fulfilled, wherein the termination condition describes the attainment of a predetermined setpoint pressure by the force value in relation to the reference surface value.

It is proposed, with regard to the threshold value, to no longer take account of the force itself or a gradient, but instead to employ individual characteristics of the examination object, in particular the breast, in order to achieve a setpoint pressure on the examination object, in particular the breast, which is generally useful for imaging but still sufficiently comfortable. To this end, it is proposed to estimate the contact surface to which the setpoint pressure is to be applied by employing the measured data acquired during automatic operation of the compression paddle. Once this contact surface has been estimated as the reference surface value, it is possible to determine the currently applied pressure from the currently applied compression force assuming this contact surface or to determine a process-specific threshold value from the setpoint pressure in order to be able to regulate to the setpoint pressure.

Specific possibilities of one or more example embodiments can therefore provide that, to check fulfillment of the termination condition,
a current pressure value which describes the current pressure on the examination object is determined from the current force value and the reference surface value, and
the current pressure value is compared with the threshold value provided as the setpoint pressure, wherein the termination condition is fulfilled if the pressure value is greater than or equal to the threshold value, or
a process-specific threshold value for the force value is determined from the setpoint pressure and the reference surface value, and
the current force value is compared with the threshold value, wherein the termination condition is fulfilled if the force value is greater than or equal to the threshold value.

In other words, it is possible on the one hand to convert the force value to a corresponding pressure value, which can be compared with the threshold value provided as the setpoint pressure, and on the other hand to provide a finally individualized threshold value for the force value determined from the setpoint pressure. In this way, it is therefore possible to take specific account of the examination object, in particular the breast, to be acquired in the current acquisition process and thus to achieve greater comfort and a high image quality. In other words, an individualization of the known procedure is proposed which takes into consideration at least the size of the examination object, in particular therefore the breast size. In this way, the acceptance of automatic adjustment processes for the compression device can be increased, enabling their use to become more widespread.

In this regard, the method can be implemented without problems on commonly used mammography devices, most of which in any case have the at least one force detection means and the position detection means. Here, the position detection means can be a sensor configured to track the movement of the compression paddle and thus also its current position tracked. Here, the object holder can be formed by the X-ray detector of the mammography device or at least comprise the X-ray detector. The associated X-ray source, which completes the acquisition arrangement, can be arranged such that it irradiates the compression paddle. Here, the mammography device can be embodied both for two-dimensional and also for three-dimensional imaging, in particular for tomosynthesis.

The use of the position detection means and the at least one force detection means makes it possible to dispense with further detection means for detecting the size of the examination object. In particular, the method described here does not require the use of any cameras or sensors which provide cause for concern or are otherwise of relevance under data protection law.

It is provided that a suitable setpoint pressure is predetermined, which is used in the termination criterion. While it is in principle conceivable in this regard to employ a general compromise for different use cases, therefore to use a fixedly predetermined setpoint pressure for different use cases, it is preferred according to one or more example embodiments that adaptations are made to the individual acquisition process, also with respect to the setpoint pressure, in order to achieve a further improvement in image quality and comfort.

A preferred development of one or more example embodiments therefore provides that the setpoint pressure is predetermined as a function of at least one item of examination information describing the examination process, in particular the time dimensionality of the image dataset to be determined. For example, it is possible to take into consideration the specific modality as well as settings of the acquisition arrangement for the acquisition process, in particular as regards the X-ray dose. In a particularly advantageous development of one or more example embodiments, it is however provided that the setpoint pressure for a three-dimensional acquisition is predetermined to be lower than for a two-dimensional acquisition. Studies have shown that lower compression can be used for three-dimensional acquisition processes, in particular tomosynthesis, without any loss of diagnostic performance. The reason for this is that image data is obtained from different directions and then combined in the reconstruction, resulting in a more robust determination of the final image dataset. It is now proposed that this be taken into consideration in order to permit more comfort for the respective patient while maintaining the necessary image quality, in particular with regard to diagnostic suitability.

In particularly advantageous exemplary embodiments, it can also be provided that at least one item of characteristic information of the examination object is determined as a function of the course of the force value over the position value, and the setpoint pressure is predetermined as a function of the characteristic information, in particular in accordance with a first assignment rule. Here, it is particularly advantageously possible to include the reference surface value as well as the course when determining the characteristic information. In other words, the measurement of the change in force over the change in position (change in travel) can give feedback about the elastic characteristics of the examination object, in particular the breast, and thus about the composition thereof. This also applies in particular if the known breast surface, in other words the reference surface value, is included. For example, conclusions can then be drawn about the composition of a breast to be acquired, for example as the ratio of glandular tissue to adipose tissue, and the setpoint pressure can be selected as the target pressure for the individual breast composition. Specifically, a first assignment rule can be used to determine a suitable setpoint pressure based on the course and preferably also the reference surface value.

The first assignment rule is preferably a look-up table (LUT). If examination information is also taken into consideration, different look-up tables can be provided for different items of examination information, for example one for two-dimensional acquisitions and one for three-dimensional acquisitions. It is however particularly preferred that provision be made for a common look-up table for the examination information and the characteristic information in order to be able to determine the suitable setpoint pressure in a simple manner.

A preferred embodiment of the present invention can provide that the first contact time instant of the compression paddle with the examination object is detected via at least one of the at least one force detection means, wherein a reference height value of the examination object is determined from the position value at the reference time instant as the distance from the object holder, and the reference surface value is determined from the reference height value. In the case of a breast, the reference height value thus determined can be construed as an initial breast thickness at the start of the compression. Here, at least one of the at least one force detection means, in particular a force sensor, can be used to detect that the compression paddle is in contact with the examination object, as said examination object naturally opposes the compression with a resistance, which is expressed in an increase in force. A favorable detection value for detecting the first contact time instant has proven to be for example five newtons. It is known on the basis of the position value where the compression paddle is located relative to the object holder on which the examination object likewise rests, so that the distance from compression paddle to object holder corresponds to the reference height value, in other words the initial examination object thickness.

For specifically determining the reference surface value from the reference height value, it can then be provided that a statistically determined second assignment rule, which assigns a statistically most probable reference surface to a reference height value as the reference surface value, in particular a look-up table, is used. In other words, the reference height value can be used as the basis for identifying a statistically typical contact surface of the examination object with the compression paddle in the target compression setting. Here, examination series, therefore trials, can for example be carried out in which the reference height value and an assigned reference surface value are determined in the manner described. There are various possibilities for determining the reference surface value for statistical measurement series from which the second assignment rule can be derived, which can be based for example at least partly on the assumption that the volume of the examination object, in particular the breast, remains constant. Clinical studies in which a camera is directed toward the breast can be used, for example. It is also possible to evaluate image data acquired with the mammography device in order to determine the contact surface of the breast (or other examination object) with the compression paddle, in particular assuming a constant volume. Such measurement series can be evaluated statistically in order to determine which contact surface is most probable as the reference surface value for a certain reference height value, in other words a certain initial examination object thickness. This most probable contact surface can then be assumed in order to process the setpoint pressure in a suitable manner. The use of such statistical analyses based on the detection of the first contact time instant, at which therefore a first contact surface with the examination object is established, which detection is possible in a simple manner and in most cases has already been carried out, enables the breast size in the form of the reference surface value to be estimated with little outlay.

Provision can also be made within the scope of the present invention, as has already been proposed in principle in the prior art, for the actuation of the actuator to take place in two operating phases, wherein in the first operating phase the compression paddle is advanced toward the examination object, in particular at a constant speed, and from the reference time instant onward in a second operating phase is moved further to compress the examination object with a compression force which in particular increases over time. In this case, the detection of the first contact time instant therefore serves a dual purpose, namely on the one hand the phase transition in the actuation of the movement of the compression paddle, and on the other hand the use as the reference time instant at which the reference height value is determined.

With regard to the force detection means, it can be provided that at least one of the at least one force detection means as a force sensor directly measures the compression force exerted via the compression paddle and/or at least one of the at least one force detection means measures a time, in particular from a first contact time instant onward and/or the second operating phase and the force value is determined on the basis of a known actuation course over time. In the first-mentioned case, the compression force exerted by the compression paddle is therefore detected directly and in a particularly reliable manner via the force sensor. The second case can however also be used (further) within the scope of one or more example embodiments if there is an actuation course which shows how the compression force is increased over time, in particular in the second operating phase. Mammography devices are already known in the prior art in which the compression force increases linearly over time in the second operating phase so that the time is therefore proportionate to the compression force.

In a particularly preferred embodiment of the present invention, it can be provided that the mammography device further has a display device, which is actuated by the control device for visualizing the determined reference surface value in relation to the size of the compression paddle and/or the object holder. This is based on the idea that the determination of the reference surface value is merely an estimate, especially if a statistical analysis is employed as the basis. This means that there is an individual tendency for errors. It is therefore proposed that the breast size used as the basis for the automatic adjustment, in other words the reference surface value, be visualized for an operator. This preferably takes place directly when said reference surface value is determined, for example at the first contact time instant. By observing the real situation, the operator can identify quickly whether the estimate is correct, and can intervene if necessary and/or make further manual corrections once the target compression setting has been reached.

Specifically, it can be provided for example that, for visualization purposes, a rectangle indicating the size of the compression paddle and/or of the object holder corresponding to the shape of the examination object is filled in partially, indicating the relative portion of the reference surface value, in particular following at least roughly the shape of the examination object and/or starting from one side of the rectangle. This produces an extremely intuitive representation, which can be set directly in relation to what is actually happening on the object holder.

Furthermore, it is particularly expedient if, in particular for a manual adjustment process, an item of information is also shown which describes how the current compression could be adapted for smaller and/or larger examination objects in order to achieve the setpoint pressure. In other words, it is additionally possible to output an indication as to whether the compression force and thus the pressure should be increased or lowered for a larger/smaller breast size or general examination object size. In this way, in addition to the intuitive rendering of the actual situation upon detection of a deviation from reality, the possibility is provided to intervene manually in the correct manner.

In a development of one or more example embodiments, it can further be provided that the control device is further embodied to visualize, for the current force value and/or at least one reference force, the relative surface of the examination object to the surface of the compression paddle for which the setpoint pressure is given. It is therefore possible, in particular in the case of manual adjustment of the compression force, to visualize the contact surface which would be ideally suited to the current compression force from the perspective of the compression, in other words with regard to the setpoint pressure. An increase in the current compression force would thus result in the contact surface visualized for example as the examination object, in particular the breast, becoming larger. The operator is thus given patient-specific feedback enabling an improved manual adjustment of the compression force.

This visualization during manual adjustment of the compression force can also be useful independently of the above-described control of the automatic adjustment, in particular using the reference surface value. A method of the type cited in the introduction would then result, wherein to support the manual adjustment of a target compression a reference contact surface is determined from the current force value and a predetermined setpoint pressure and the size of the reference contact surface relative to the surface of the compression paddle is visualized on a display device of the mammography device. It is furthermore also conceivable in this connection to provide a general, permanent visualization for a minimum value of the force value and a maximum value of the force value and, if necessary, at least one intermediate value on a scale indicating the current force value. Here, the display concepts already cited above, in particular the rectangle symbolizing the compression paddle which is filled in progressively by the breast as the size increases, can be used.

In addition to the method, one or more example embodiments also relates to a mammography device, having:
an object holder for an examination object to be acquired, in particular a breast,
a compression device assigned to the object holder with at least one movable compression paddle, to which an actuator for moving the compression paddle is assigned,
at least one position detection means for detecting a position value which describes the current position of the movable compression paddle,
at least one force detection means for detecting a force value which describes the force exerted by the compression paddle, and
a control device, which is embodied for automatically adjusting a target compression setting of the movable compression paddle for an examination object by actuating the actuator as a function of the position value, the force value and a predetermined threshold value, wherein for automatically adjusting a compression of the examination object for an acquisition process, the control device has:
an interface for receiving the force value and the position value,
a determination unit for determining a reference surface value which describes an assumed contact surface between the compressed examination object and the movable compression paddle in the target compression setting, using the force value and the position value at at least one reference time instant, and
a control unit for compressing the examination object by actuating the actuator until a termination condition is fulfilled, wherein the termination condition describes the attainment of a predetermined target pressure by the force value in relation to the reference surface value.

All the statements relating to the method according to one or more example embodiments can be applied analogously to the mammography device according one or more example embodiments, and vice versa, and therefore the aforementioned advantages can likewise be achieved by the mammography device.

The control device can be embodied specifically to carry out a method according one or more example embodiments. It has at least one processor and at least one storage means (e.g., storage device), wherein for example the look-up tables (first and second assignment rule) already described can be stored in the storage means. Functional units are provided by hardware and/or software in order to control the operation of the mammography device in general and in particular to be able to carry out steps of the method according to one or more example embodiments. Here, in addition to the determination unit already cited and the control unit, further functional units can also be provided in order to implement further embodiments of the method. For example, the control device can also have a specification unit in order to determine the setpoint pressure for a respective use case and patient, in particular using the first assignment rule. A trigger unit can also be provided in order to detect the first contact time instant as the reference time instant. Provision can also be made for a representation unit for actuating the display device, in particular for visualizing the reference surface value and/or the reference contact surface during manual adjustment.

The method according to one or more example embodiments can also be implemented as a computer program. The computer program is embodied such that, when the computer program is executed in a control device of a mammography device, said mammography device is caused to carry out the steps of a method according to one or more example embodiments. The computer program can be stored on an electronically readable data storage medium, which therefore comprises control information stored thereon, which control information comprises at least one computer program according to one or more example embodiments and is embodied such that when the data storage medium is used in a control device of a mammography device, said mammography device is embodied to carry out a method according to one or more example embodiments.

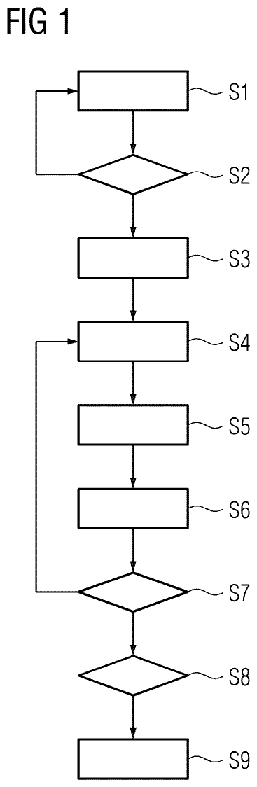

FIG. 1 shows a flow diagram of an exemplary embodiment of the method according to the invention, as it can be carried out on a control device of a mammography device for automatically adjusting a compression of an examination object, in this case a breast, which corresponds to a setpoint pressure onto the breast. To this end, the mammography device has, as is known in principle, an object holder which can comprise the X-ray detector or be formed thereby. Toward the X-ray emitter of the mammography device, the object holder is followed by a compression paddle of a compression device, which can be moved via an actuator, which can be actuated by the control device, in order to exert compression onto the breast disposed between the object holder and the compression paddle.

In a step S1, a first operating phase for adjusting the target compression setting begins, in which the compression paddle, which was initially still spaced apart from the breast, is advanced toward the breast. This can take place at a constant speed. In a step S2, the measured data of a first force detection means, for example a force sensor, is used to check whether a first contact time instant has been reached in which the compression paddle touches the breast and establishes a first contact surface. The detected first contact time instant also forms a reference time instant.

As is known in principle, the compression paddle, in particular assigned to the actuator or as part of the actuator, is assigned a position detection means, the position value of which also indicates the spacing between the object holder and the compression paddle. The position detection means is now used in a step S3 to determine a reference height value of the breast as the distance between the compression paddle and the object holder at the reference time instant, in other words the first contact time instant. The reference height value is in turn used to determine therefrom, via a second assignment rule, in particular a look-up table, a statistically most probable contact surface of the compression paddle with the breast in the target compression setting. To this end, the second assignment rule was determined by a statistical analysis of corresponding measured data, in which height values with assigned contact surfaces, which are calculated for example assuming constant volume, are determined. This statistically most probable contact surface forms the reference surface value.

In a step S4, a second operating phase of the compression paddle then begins, in which said compression paddle is moved with increasing compression force in order to compress the breast. Here, a force value describing the compression force currently being exerted onto the breast is also determined via the first or a second force detection means. Therein, a force sensor can in turn be used, which can be integrated for example in the compression paddle. It is however also possible to specify a fixed course of the compression force over time for the second operating phase and to measure the time since the first contact time instant, so that the second force detection means can be a timer which supplies the force value via the fixed course.

In a step S5, a setpoint pressure is determined at which there is a compression of the breast which permits sufficient image quality for the impending acquisition process but is still comfortable for the patient. Alongside a less preferred, general specification of the setpoint pressure, it is also possible to select said setpoint pressure as a function of an item of examination information of the examination process and/or an item of characteristic information of the breast, which can be derived from the course of the force value. If the setpoint pressure is dependent only on an item of examination information, for example whether two-dimensional or three-dimensional imaging is to take place, the step S5 can also be carried out earlier during the course of the method. In the case of three-dimensional imaging, for example tomosynthesis, the setpoint pressure, in other words the compression, can be selected to be lower.

It is however particularly preferred that an item of characteristic information be included. The measurement of the change in compression force over the change in position (based on the force value and the position value) contains—given a known reference contact surface—an item of information about the elastic characteristics of the breast and thus about the composition of the breast, in particular the proportion of glandular tissue relative to adipose tissue. By taking such individual characteristics of the breast into consideration, it is possible to select the setpoint pressure in an excellent manner such that no excessive discomfort or even pain occurs as a result of the compression. A first assignment rule, preferably in turn a look-up table, is used in order to assign a setpoint pressure to the examination information and the characteristic information. Here, the setpoint pressure can be updated constantly during the second operating phase as the characteristic information is constantly updated or supplemented.

In a step S6, to support an operator monitoring the automatic adjustment, the reference contact surface is visualized on a display device of the mammography device. This is shown relative to the surface of the compression paddle and/or of the object holder, so that the operator can estimate whether the reference contact surface has been estimated correctly on the basis of the statistically most probable selection or whether there is an individual deviation case. In the event of a strong deviation, the automatic adjustment can be terminated and the adjustment continued manually, as described in more detail further below. However, the display of step S5 is maintained even once the target compression setting has been reached in order to enable readjustments to be made as easily as possible.

Because the setpoint pressure and the reference contact surface are known via the reference surface value, it is possible to read off from the force value whether the setpoint pressure and thus the target compression setting has been reached. This is checked in a termination condition in step S7. Here, it is possible on the one hand to determine a current pressure value from the current force value and the reference surface value and compare it with the setpoint pressure and, on the other hand, to derive an individual threshold value for the force value from the setpoint pressure and the reference surface value and to monitor whether this is reached. The second operating phase (step S4) is continued for as long as the termination condition is not fulfilled.

If the termination condition is fulfilled, however, in step S8—while the relative size of the breast according to the reference surface value to the object holder and/or to the compression paddle continues to be displayed—a check is made to determine whether a manual readjustment is required, for example if the breast size differs too much from the statistically most likely assumption. Then, in a step S9, the compression force/compression setting can be adjusted manually, wherein an adjustment aid is output on the display device in parallel.

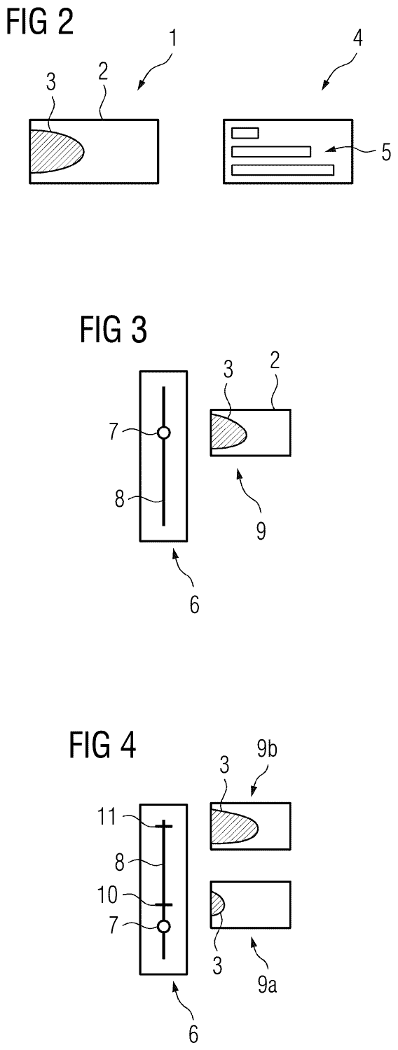

This will be explained in more detail with respect to FIG. 2, which shows a first possible display on the display device. A first display element 1 shows, as a rectangle 2 which is filled from one side with a stylized breast graphic 3, the contact surface according to the reference surface value relative to the compression paddle and/or the object holder symbolized by the rectangle 2. The display element 1 is displayed continuously during the second operating phase, therefore from determination in step S3. If a manual adjustment of the compression setting is required (cf. step S8 and step S9), an item of information 5 is shown as a further display element which describes how the current compression could be adapted for smaller and/or larger examination objects in order to achieve the setpoint pressure.

A display concept according to the display element 1 is also expedient in other respects during the manual adjustment of a suitable compression setting. FIG. 3 therefore shows, in a second conceivable display on the display device, a display element 6 which shows the current compression force (marker 7 on a scale 8). In addition to the marker 7, the breast size (contact surface) resulting from this current compression force is now represented as a further display element 9. The operator performing the adjustment is thus informed in an intuitively comprehensible manner as to whether a suitable compression setting has been found. During the manual selection of a compression setting, the control device thus visualizes, for the current force value, the relative surface of the breast to the surface of the compression paddle/object holder for which the setpoint pressure is given.

As FIG. 4 shows, this is also possible as a static information display. There, a minimum value 10 and a maximum value 11 are marked on the scale 8, to which in each case a display element 9a, 9b with correspondingly determined breast graphics 3 is assigned. It is also possible if necessary to show corresponding display elements for further reference forces in addition to the minimum value 10 and the maximum value 11.

FIG. 5 shows a schematic outline of a mammography device 12 according to one or more example embodiments of the invention. This has a stand 13, in which a holder unit 14 comprising the acquisition arrangement and the holder arrangement of the mammography device 12 is guided in a height-adjustable manner. The holder unit therefore comprises an object holder 15, which is formed by the X-ray detector 16 or at least comprises the same. The X-ray detector 16 can receive X-ray radiation of an X-ray emitter 17 which is in particular supported movably for tomosynthesis purposes. A compression paddle 18 of a compression device is arranged movably between the X-ray emitter 17 and the object holder 15/X-ray detector 16. A compressed breast 19 between the object holder 15 and the compression paddle 18 is also indicated schematically in order to illustrate the operating principle.

The compression paddle 18 can be moved automatically via an actuator 20. In addition, a position detection means 21 as well as at least one force detection means 22 are also assigned to the compression paddle 18.

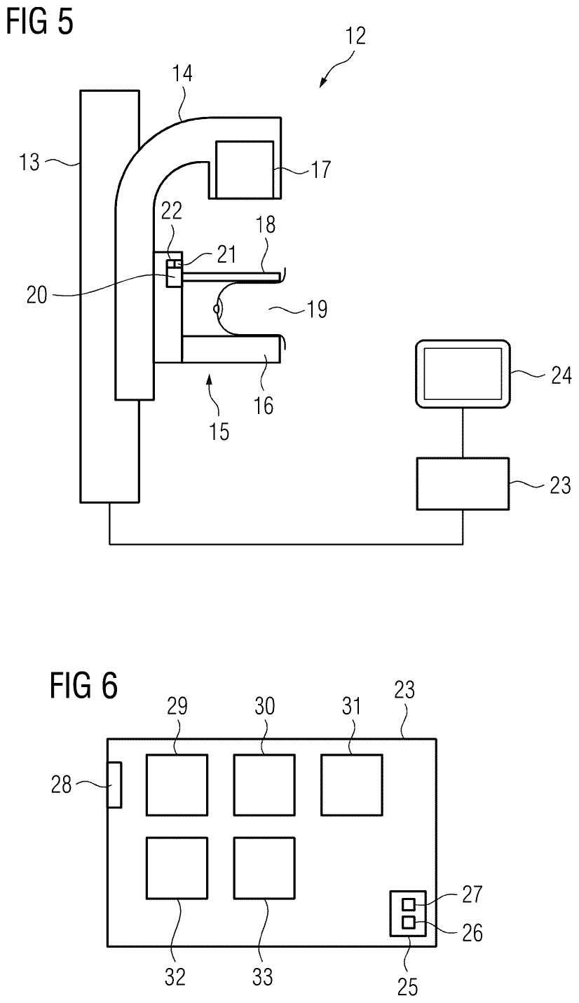

The operation of the mammography device 12 is controlled by way of a control device 23, which is also connected to a display device 24. The control device 23 is embodied to carry out the method explained with respect to FIG. 1. To this end, the control device 23 has the functional structure shown in more detail in FIG. 6.

For the sake of clarity, FIG. 6 only shows the components which are relevant to the method, wherein the control device 23 can of course also comprise further functional units, for example an acquisition unit for controlling acquisition operation.

The control device 23 comprises a storage means 25, in which here in particular the first assignment rule 26 and the second assignment rule 27 are stored, both as a look-up table. Among other data, a current position value of the position detection means 21 and a current force value of the force detection means 22 are received via an interface 28. A control unit 29 controls the movement of or force exerted by the compression paddle 18 via the actuator 20, in particular according to step S1 in the first operating phase and according to step S4 in the second operating phase. A trigger unit 30 monitors according to step S2 whether the first contact time instant has been reached. A detection unit 31 is embodied to detect the reference surface value from the reference height value according to step S3 using the second assignment rule 27.

In a specification unit 32, the setpoint pressure can be detected according to step S5, for which, as described, the first assignment rule 26 is used. Finally, provision is also made for a display unit 33 for generating the various displays, in particular according to FIG. 2 to FIG. 4, cf. also steps S6 and S9.

The control unit 29 is also responsible for monitoring the termination condition here (step S7). During the manual adjustment of a compression setting, in particular also during an adjustment starting from a target compression setting, a user interaction unit (not shown), which can comprise the display unit 33, can receive and process user inputs, in particular such that the control unit 29 can actuate the actuator 20 accordingly.

Independent of the grammatical term usage, individuals with male, female or other gender identities are included within the term.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors s used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility (also referred to as a data processing facility) or computer device to execute the program of any of the above mentioned embodiments and/or to perform the method of any of the above mentioned embodiments.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the invention has been illustrated and described in detail by the preferred exemplary embodiments, it is not limited by the disclosed examples and a person skilled in the art can derive other variation herefrom without departing from the protective scope of the invention.

1. A computer-implemented method for operating a mammography device and for automatically adjusting a compression of an examination object for an acquisition process, the mammography device including an object holder for the examination object to be acquired, a compression device assigned to the object holder with at least one movable compression paddle, to which an actuator for moving the compression paddle is assigned, at least one position detector configured to detect a position value which describes a current position of the movable compression paddle, and at least one force detector configured to detect a force value which describes the force exerted by the compression paddle, and a control device configured to automatically adjust a target compression setting of the movable compression paddle for an examination object by actuating the actuator as a function of the position value, the force value and a predetermined threshold value,
the method comprising:
determining a reference surface value which describes an assumed contact surface between the compressed examination object and the movable compression paddle in the target compression setting, using the force value and the position value at at least one reference time instant; and
compressing the examination object by actuating the actuator until a termination condition is fulfilled, wherein the termination condition describes an attainment of a predetermined setpoint pressure by the force value in relation to the reference surface value.

2. The method of claim 1, wherein, in order to check the fulfillment of the termination condition,
(i) a current pressure value which describes the current pressure on the examination object is determined from the current force value and the reference surface value, and
the current pressure value is compared with the threshold value provided as the setpoint pressure, wherein the termination condition is fulfilled if the pressure value is greater than or equal to the threshold value,
or
(ii) a process-specific threshold value for the force value is determined from the setpoint pressure and the reference surface value, and
the current force value is compared with the threshold value, wherein the termination condition is fulfilled if the force value is greater than or equal to the threshold value.

3. The method of claim 1, wherein the setpoint pressure is predetermined as a function of at least one item of examination information describing the examination process.

4. The method of claim 3, wherein the setpoint pressure for a three-dimensional acquisition is predetermined to be lower than for a two-dimensional acquisition.

5. The method of claim 1, wherein at least one item of characteristic information of the examination object is determined as a function of a course of the force value over the position value, and the setpoint pressure is predetermined as a function of the characteristic information.

6. The method of claim 5, wherein the reference surface value and the course are included when determining the characteristic information.

7. The method of claim 1, wherein a first contact time instant of the compression paddle with the examination object is detected via at least one of the at least one force detector, wherein a reference height value of the examination object is determined from the position value at the reference time instant as a distance from the object holder, and the reference surface value is determined from the reference height value.

8. The method of claim 7, wherein, the determining the reference surface value from the reference height value uses a statistically determined second assignment rule which assigns a statistically most probable reference surface to a reference height value as the reference surface value.

9. The method of claim 7, wherein the actuating the actuator takes place in two operating phases, wherein in the first operating phase the compression paddle is advanced toward the examination object, and from the reference time instant onward in a second operating phase the compression paddle is moved further to compress the examination object with a compression force that increases over time.

10. The method of claim 9, wherein (i) at least one of the at least one force detector is a force sensor that directly measures the compression force exerted via the compression paddle, (ii) at least one of the at least one force detection means measures a time, or (iii) the second operating phase and the force value is determined on the basis of a known actuation course over time.

11. The method of claim 1, wherein the mammography device includes a display device, which is actuated by the control device for visualizing the determined reference surface value in relation to a size of at least one of the compression paddle or the object holder.

12. The method of claim 11, wherein, for visualization purposes, a rectangle indicating a size of at least one of the compression paddle or of the object holder corresponding to the shape of the examination object is filled in partially, indicating the relative portion of the reference surface value.

13. The method of claim 12, wherein, an item of information is shown which describes an adapted current compression to achieve the setpoint pressure.

14. The method of claim 11, wherein the control device is configured to visualize, for at least one of the current force value or at least one reference force, for which relative surface of the examination object to the surface of the compression paddle the setpoint pressure is given.

15. A mammography device, comprising:
an object holder for an examination object to be acquired;
a compression device assigned to the object holder with at least one movable compression paddle, to which an actuator for moving the compression paddle is assigned;
at least one position detector configured to detect a position value which describes a current position of the movable compression paddle, and at least one force detector configured to detect a force value which describes the force exerted by the compression paddle; and
a control device configured to automatically adjust a target compression setting of the movable compression paddle for an examination object by actuating the actuator as a function of the position value, the force value and a predetermined threshold value,
wherein for automatically adjusting a compression of the examination object for an acquisition process, the control device includes,
an interface configured to receive the force value and the position value,
a determination unit configured to determine a reference surface value which describes an assumed contact surface between the compressed examination object and the movable compression paddle in the target compression setting, using the force value and the position value at at least one reference time instant, and
a control unit configured to compress the examination object by actuating the actuator until a termination condition is fulfilled, wherein the termination condition describes an attainment of a predetermined setpoint pressure by the force value in relation to the reference surface value.

16. The method of claim 2, wherein the setpoint pressure is predetermined as a function of at least one item of examination information describing the examination process.

17. The method of claim 16, wherein the setpoint pressure for a three-dimensional acquisition is predetermined to be lower than for a two-dimensional acquisition.

18. The method of claim 2, wherein at least one item of characteristic information of the examination object is determined as a function of a course of the force value over the position value, and the setpoint pressure is predetermined as a function of the characteristic information.

19. The method of claim 18, wherein the reference surface value and the course are included when determining the characteristic information.

20. The method of claim 10, wherein the at least one of the at least one force detection means measures the time from a first contact onward.
